(19) United States
(12) Patent Application Publication
(10) Pub. No.: US 2010/0048441 A1
(43) Pub. Date: Feb. 25, 2010

(54) Multisensory Fragrance Compositions

(75) Inventors: John Martin Behan, Ashford (GB); Anne Churchill, Canterbury (GB); Keith Douglas Perring, Ashford (GB); Andrew Perfect, Naarden (NL)

Correspondence Address: PARFOMAK, ANDREW N.;NORRIS MCLAUGHLIN & MARCUS PA, 875 THIRD AVE, 8TH FLOOR, NEW YORK, NY, 10022, US

(73) Assignee: Givaudan Nederland Services, Naarden (NL)

(21) Appl. No.: 12/529,418

(22) PCT Filed: Mar. 3, 2008
(86) PCT No.: PCT/EP2008/001650

§ 371 Date: Oct. 21, 2009

(30) Foreign Application Priority Data
Mar. 1, 2007 (DE) 102007010009.6

(51) Int. Cl. C11D 3/50; A61K 8/34; A61K 8/35; A61K 8/37; A61K 8/49; A61Q 13/00

(52) U.S. Cl. 510/103; 512/8; 512/12; 512/27; 512/21; 512/25; 512/23; 512/4; 510/102; 510/107; 510/101

(57) ABSTRACT

The present invention provides perfume compositions that control the perception of tactile attributes of consumer products that contain the inventive compositions and/or the surfaces of articles treated therewith. The perfume compositions comprise from about 10% by weight

### FIELD OF THE INVENTION

The present invention relates to fragrance compositions. For the purposes of this invention a fragrance composition is defined as a mixture of fragrance ingredients, if desired mixed with or dissolved in a suitable solvent or solvents and/or mixed with a solid substrate. In this specification, the terms perfume and fragrance are used synonymously. Perfume or fragrance ingredients are well known to those skilled in the art, and include those mentioned, for example, in S. Arctander, Perfume and Flavor Chemicals (Montclair, N.J., 1969), in S. Arctander, Perfume and Flavor Materials of Natural Origin (Elizabeth, N.J., 1960) and in “Flavor and Fragrance Materials—1991”, Allured Publishing Co. Wheaton, Ill. USA. Perfume ingredients may include natural products such as extracts, essential oils, absolutes, resinoids, resins, concretes etc., and also synthetic basic substances such as hydrocarbons, alcohols, aldehydes, ketones, ethers, acids, esters, acetals, ketals, nitrites, etc., including saturated and unsaturated compounds, aliphatic, alicyclic and heterocyclic compounds.

In particular, the present invention relates to fragrance compositions that enhance positive tactile perceptions of surfaces treated with such compositions. The invention also concerns the use of fragrance compositions to enhance the positive tactile perceptions of the texture or feel and/or reduces the negative tactile perceptions of consumer products containing the inventive compositions, such as shampoos or skin creams, hard surface cleaners and laundry products.

### BACKGROUND OF THE INVENTION

There are many consumer goods available to satisfy a wide variety of consumer desires. Consumers expect, for example, laundry powders to remove dirt and stains from fabrics, and underarm deodorants and air freshening products to release agreeable odours and reduce the perception of unpleasant odours.

In general, the functional qualities of products are considered to be determined by perceptions associated with a limited combination of the five human senses. For example, a fabric may look clean after using a laundry powder, an underarm may smell fresh after using a deodorant, and skin may feel soft after using a moisturising cream. The separate senses of vision, smell and touch are associated in these cases with perceived qualities. However, research carried out in the USA suggests that the smell of toiletry products is more important to the consumer than the actual function of the product (J. Byrne-Quinn, Perfume, people, perceptions and products. in S. Van Toller & G. Dodd (Eds.), Perfumery: The Psychology and Biology of fragrance (pp. 205-216). (1988) New York: Chapman and Hall.). Additional research at Oxford University has shown that an individual's senses do not operate in complete isolation and that the perceptions of one sense may be enhanced by another. For example, a person who is short sighted may find their ability to hear in a noisy environment is enhanced or diminished dependent on whether or not they are wearing spectacles or contact lenses. It has been demonstrated also that while most people perceive cherry flavoured and cherry coloured drinks to taste of cherry, many people considered the same drink to taste of lime when coloured green, and some thought it tasted of orange when coloured orange (Du Bose. Journal of Food Science, (1980) 45, 1393-1399).

It has been found that the sense of touch may be altered by auditory stimuli. This was illustrated by the “parchment skin illusion” (Jousmäki and Hari (Current Biology, (1998) 8, 869-872.)), wherein it was reported that individuals could be made to perceive that their hands were smoother or rougher simply by manipulating the sounds introduced to them when they rubbed their hands together. A study by Dematte et al (Chemical Senses (2006), 31, 531-538) demonstrates that the presence of an odour can modify the tactile perception of, for example, fabric softness. This study puts forward the propositions that tactile perception may be modified by odour through (i) an associative effect caused by repeat exposure to odour/tactile combinations; (ii) pleasant odours making the perception of texture more agreeable (e.g. softer), or (iii) the presence of a pleasant odour inducing a general change in mood that is reflected in response to, for example, rating scores for softness.

While these studies have demonstrated that specific sensorial effects can be obtained using targeted combinations of stimuli, there is little or nothing in the prior art that teaches systematic means of formulating fragrances to obtain a multi-sensory effect. Furthermore there is little or nothing to suggest that a fragrance introduced as one part of a complete product package in an in-use situation can be utilized to modify the sensory perception of attributes not normally associated with fragrance (e.g. touch and feel sensations) of the said product or substrates treated therewith.

### SUMMARY OF THE INVENTION

The present invention relates to fragrance compositions that influence or enhance the senses of touch. Furthermore, the current invention involves the use of fragrance compositions that enhance the perception of a range of touch attributes to maximise the functional properties of consumer products.

In particular, the present invention is directed to a method for enhancing the perception of tactile characteristics through the olefactive perception of a fragrance composition delivered either through use of a fragranced product or by direct inhalation. Specifically, the method involves delivering an effective amount of a fragrance composition, preferably one with positive hedonics, to cause an individual's perception of tactile sensations to be statistically significantly different to that obtained in the presence of prior art fragrance that is not designed according to the present invention to obtain multi-sensory effects.

For the purposes of this invention a fragrance composition is defined as a mixture of fragrance ingredients, if desired mixed with or dissolved in a suitable solvent or solvents and/or mixed with a solid substrate, which can stimulate olfactory and/or trigeminal chemoreceptors in the nasal cavity and cause a physiological or psychological response. In this specification, the terms perfume and fragrance are used synonymously. Preferred fragrance compositions are those that bring about a hedonically positive response, where a hedonically positive fragrance composition is one having a scent to which an individual has a pleasant or positive reaction, also termed a positive hedonic response.

In the design and manufacture of consumer products it is desirable to maximise the functional properties of the product. However, physical modification of product ingredients may introduce undesirable properties. For instance, additives may be included in hair products, such as shampoos and conditioners, to enhance hair softness and manageability, but excessive use of additives may results in undesirable effects such as heaviness and/or greasiness.

Fragrance compositions are described that influence the perception of various different attributes using a sense other than olfaction. By influencing, for example, perception of attributes such as manageability, smoothness, softness, silkiness, sleekness, conditioning, dryness, coarseness, tangled, sliminess, stickiness, or brittleness, fragrance compositions arising from this invention provide a broad sensual appeal and permit consumers to better appreciate products and materials.

In a first aspect of the invention, a perfume composition comprises:
a) from about 10% by weight in toto of at least two ingredients selected from group A and
b) from around 22% by weight in toto of at least five ingredients selected from group B, wherein
   i. Group A consists of:
      2-ethyl-4-(2,2,3-trimethylcyclopent-3-en-1-yl)but-2-en-1-ol, 2H-chromen-2-one, Cyclopentadecanolide, 1,4-Dioxacycloheptadecane-5,-17-dione, Ethyl Vanillin, oxacyclohexadec-12(13)-en-2-one, 1,3-benzodioxole-5-carbaldehyde and methyl(3-oxo-2-pentylcyclopentyl)acetate and
   ii. Group B consists of:
      3a,6,6,9a-tetramethyldodecahydronaphtho[2,1-{b}]furan, Amyl Salicylate, 4-(methyloxy)benzaldehyde, Benzyl Acetate, 3-[4-(1,1-dimethylethyl)phenyl]propanal, Cis-3-Hexenyl Acetate, Citronellyl Acetate, Cyclodecalactone-Gamma, 4-methyl-2-(2-methylpropyl)tetrahydro-2H-pyran-4-ol, Geraniol, Geranium Oil, Hexyl Salicylate, 2-(methyloxy)-4-[prop-1-enyl]phenyl acetate, 3-[4-(1,1-dimethylethyl)phenyl]-2-methylpropanal, Linalool, 4-(4-hydroxy-4-methylpentyl)cyclohex-3-ene-1-carbaldehyde, [4-(1-methylethyl)cyclohexyl]methanol, 3-methyl-4-(2,6,6-trimethylcyclohex-2-en-1-yl)but-3-en-2-one, Phenyl Ethyl Acetate, Phenyl Ethyl Alcohol and Tetrahydrolinalool.

Unless otherwise specified, all percentages are based on the total weight of fragrance ingredients constituting the perfume composition, thus excluding any non-odourous or very low odour solvents or vehicles.

“Non-odourous or very low odour” means that in scores less than 80 on an odour index scale as set out in EP0404470 (based on comparison with the odour intensity of a control sample of a 10% solution of benzyl acetate in dipropylene glycol, which corresponds to an index of 100), and includes diethylphthelate, dipropylene glycol, triacetin, benzyl benzoate, triethyl citrate, Herculyn D (trade mark), isopropyl myristrate and acetyl tributylcitrate.

Another aspect of the invention relates to a method of delivering enhanced tactile perception of products containing fragrances formulated according to the invention.

A further aspect of the invention relates to a method of delivering enhanced tactile perception of surfaces treated with consumer products comprising fragrances formulated according to the invention.

It is preferred that the effect of the fragrance composition on the perception of a normosmic person who finds the fragrance composition to be hedonically positive provides a beneficial change in the perceived touch perception of consumer products containing fragrances formulated according to the invention and of the fabric/hair/skin/hard surface treated therewith.

It was found that the use of a fragranced product comprising a fragrance composition formulated according to the invention significantly alters the perception of a person using the fragranced product, of smoothness and/or softness and/or other tactile attributes. In particular, it was found that the inhalation of a hedonically positive fragrance composition could significantly alter the perception of a person such that perceived tactile properties are enhanced or suppressed in comparison with a product containing a fragrance that is not created according to the invention.

The fragrance composition is provided as a formulated blend of the essential odorants to cause the desired effect, and optionally contains additional ingredients at appropriate concentrations for hedonic properties. The fragrance composition can be administered as a constituent of a fully formulated consumer product (such as a shampoo) or in combination with carriers, preferably low odour or odourless carriers such as mineral oil or water, and additives such as preservatives and the like. Preferably, the fragrance composition provides a hedonically positive response by the inhaling individual.

Tactile perception-altering fragrance ingredients for use in compositions and methods according to the invention are selected from the forthcoming list which includes includes, where appropriate, trade names and suppliers of the materials of the invention, as well as common or trivial names used in the perfume industry. Descriptions of these materials may be found in standard perfume industry texts such as S. Arctander, Perfume and Flavor Chemicals (Montclair, N.J., 1969), in S. Arctander, Perfume and Flavor Materials of Natural Origin (Elizabeth, N.J., 1960), “Flavor and Fragrance Materials—1991”, Allured Publishing Co. Wheaton, Ill. USA; and “Common Fragrance & Flavor Materials”, VCH, Weinheim, Germany 1990, by Bauer, Garbe and Surburg, ISBN 3-527-27961:

2-ethyl-4-(2,2,3-trimethylcyclopent-3-en-1-yl)but-2-en-1-ol (e.g. Bangalol™ (Q)), Citronellol, 2H-chromen-2-one (Coumarin), Cyclopentadecanolide, 1,4-Dioxacycloheptadecane-5,-17-dione (Ethylene Brassylate), Ethyl Vanillin, oxacyclohexadec-12(13)-en-2-one (e.g. Habanolide™ (F)), 1,3-benzodioxole-5-carbaldehyde (Heliotropin) and methyl(3-oxo-2-pentylcyclopentyl)acetate (Methyl Dihydrojasmonate Super™ (Q)) odorants, and examples of group B odorants include 3a,6,6,9a-tetramethyldodecahydronaphtho[2,1-{b}]furan (e.g. Ambroxan™ (H), Ambrox™ (F), or Cetalox™ (F)), Amyl Salicylate, 4-(methyloxy)benzaldehyde (Anisic Aldehyde), Benzyl Acetate, 3-[4-(1,1-dimethylethyl)phenyl]propanal (e.g. Bourgeonal™ (Q)), Cis-3-Hexenyl Acetate, Citronellyl Acetate, Cyclodecalactone-Gamma, 4-methyl-2-(2-methylpropyl)tetrahydro-2H-pyran-4-ol (e.g. Florosa™ (Q)), Geraniol, Geranium Oil, Hexyl Salicylate, 2-(methyloxy)-4-[prop-1-enyl]phenyl acetate (Iso Eugenyl Acetate), 3-[4-(1,1-dimethylethyl)phenyl]-2-methylpropanal (Lily aldehyde e.g. Lilial™ (G)), Linalool, 4-(4-hydroxy-4-methylpentyl)cyclohex-3-ene-1-carbaldehyde (e.g. Lyral™ (IFF)), [4-(1-methylethyl)cyclohexyl]methanol (e.g. Mayol™ (F)), 3-methyl-4-(2,6,6-trimethylcyclohex-2-en-1-yl)but-3-en-2-one (e.g. Methyl Ionone Alpha Iso), Phenyl Ethyl Acetate, Phenyl Ethyl Alcohol and Tetrahydrolinalool.

F: Supplied by or trade mark of Firmenich

G: Supplied by or trade mark of Givaudan

IFF: Supplied by or trade mark of International Flavors & Fragrances

Q: Supplied by or trade mark of Quest International

The present invention is based on extensive consumer testing of fragrances and statistical analysis of the resulting data to classify the materials into different categories, namely those that are active in altering the perceptions of tactile attributes and those that are inactive (Z). The active materials were further split into a group of particularly active materials, referred to as Group A, and materials with a lesser activity, group B.

Fragrance compositions can be readily screened and assessed for positive hedonics and effectiveness in altering the perception of tactile attributes. For example, a fragrance composition can be administered to an individual who is questioned as to a positive or negative reaction to the pleasantness of the scent. The fragrance composition can then be administered to the individual for inhalation, or for them to use according to the normal procedure for using the selected product to assess its effectiveness in modifying their perception of tactile attributes.

For example, a method of screening a fragrance composition for effectively altering perception of tactile attributes can comprise the steps of: administering a supra-threshold and non-irritant concentration of a composition consisting essentially of one or more fragrance compositions to an individual for inhalation; having the individual rate a list of textural attributes (such as smoothness, softness, manageability) of, for example, fabric or hair; comparing the estimate of the tactile properties to actual properties (i.e. without fragrance present) to provide a “difference value”; and then determine the statistical significance of the difference value; and eliminating the fragrance composition as being ineffective for altering perception of fabric, hair or skin softness if not statistically significant.

A second example of a method of screening a fragrance composition for effectively altering perception of tactile attributes can comprise the steps of: providing a consumer product (such as a shampoo) containing a supra-threshold and non-irritant concentration of a composition consisting essentially of one or more fragrance compositions to an individual for use in the normal way (according to standard pack instructions for use of such a product); having the individual rate a list of textural attributes (such as smoothness, softness, manageability) of, for example, fabric or hair, during use of the product; comparing the estimate of the tactile properties to actual properties (i.e. without fragrance present) to provide a “difference value”; or comparing the estimate of the tactile properties to those obtained with another product not formulated according to this invention to provide a “difference value”; and then determine the statistical significance of the difference value; and eliminating the fragrance composition as being ineffective for altering perception of fabric, hair or skin softness if not statistically significant.

The fragrance composition may be dispensed to an individual in a form that can be used for a specific function (e.g. washing hair, or fabric, or applying to skin), that provides a vaporous emission for inhalation, or it may be applied to a surface, such as fabric or hair, in a number of forms including, for example, as a liquid, powder, gel, cream, paste, and the like. The fragrance composition can be administered in combination with low odour carrier such as mineral oil or water and/or low odour additives, and can be formulated with a viscosity effective to allow for aerosolization. The fragrance composition can be dispensed onto surfaces, for example, by direct application, or by an aerosol or non-aerosol spray, among other modes of application. The fragrance composition may be incorporated into a consumer product (such as shampoo, hair conditioner, fabric washing powder or liquid or skin cream) and used according to standard pack instructions in order to deposit fragrance onto the surface treated (e.g. during washing hair, or fabric or applying skin cream).

The effectiveness of such a fragrance composition in a real use situation can be confirmed by including the fragrance in a fully formulated product and asking a sample of the consumer population to use the product and report on its touch and feel characteristics during use and the touch or feel characteristics of their hair, skin or fabric during and after use. The fragrance should be included in the product at a level that is normal for the type of product being tested (e.g. 0.5% in shampoo base), and respondents should be instructed to use the product as they would normally use the product to under normal in-use conditions.

The composition comprises a hedonically positive fragrance composition in a supra-threshold and non-irritant concentration effective to alter perception of tactile attributes upon inhalation such that the tactile properties are perceived as being significantly different than the actual tactile properties.

The invention will be further described by reference to the following examples. These examples are not meant to limit the scope of the invention that has been set forth in the foregoing description. Variations within the concepts of the invention are apparent to those skilled in the art. The disclosures of the cited references throughout the application are incorporated by reference herein.

### EXAMPLE 1

### Consumer In-Use Test: Influence of Fragrance on Perceived Texture Attributes of Shampoo and of Hair

Experimental Design

This experiment was designed to define the fragrance characteristics that modify the perceptions of the touch and feel of a shampoo product before use and during use and the feel of the hair during use of the product and after use. Identical shampoo bases were prepared and each was perfumed with different fragrances. Before the start of the experimental work the shampoos were tested for their physical properties (viscosity and foaming) to confirm that the fragrances had not modified the physical parameters.

During the evaluation, the respondents completed a questionnaire assessing the perceived touch or feel of the product and of the hair during use. The respondents' attention was not drawn to the fragrance of the products. At no time were the respondents asked to evaluate the fragrance. The products were assessed for (i) feel on the hands before it is applied to the hair; (ii) feel on the hands and hair whilst applying it, including the lather; (iii) how the hair felt after using the product, but whilst the hair was still wet; and (iv) how the hair felt after drying but before using any styling products.

Statistical analysis of the results enabled ingredients to be sorted into active and inactive groups, with the active materials being further sorted into highly active and moderately active ingredients. The group containing highly active ingredients was labelled Group A, with the moderately active ingredients being placed in group B. Table 1 displays constituents of four active and four inactive fragrance compositions that were tested according to the protocol described above.

| Ingredient Name | Active 1 | Active 2 | Active 3 | Active 4 | Inactive 1 | Inactive 2 | Inactive 3 | Inactive 4 |
|---|---|---|---|---|---|---|---|---|
| Ambroxan | 0.3 | 0 | 0 | 0 | 0 | 0 | 0 | 0 |
| Amyl Salicylate | 0 | 0 | 1 | 0.2 | 0 | 0 | 0 | 0 |
| Anisic Aldehyde | 0 | 0 | 5 | 0 | 0 | 0 | 0 | 0 |
| Bangalol | 1.1 | 0 | 0.5 | 0.3 | 0 | 0 | 1 | 0 |
| Benzyl Acetate | 3.4 | 1.5 | 2 | 0 | 7.4 | 1.6 | 0 | 0 |
| Bourgeonal | 0.5 | 0.5 | 0 | 0 | 0 | 0 | 0 | 0 |
| Cis-3-hexenyl Acetate | 0 | 0.25 | 0 | 0.4 | 0 | 0.3 | 0.1 | 0.2 |
| Citronellol | 0 | 3.7 | 10 | 1 | 1.2 | 1.7 | 5 | 8 |
| Citronellyl Acetate | 1 | 0.7 | 0 | 0 | 0 | 0.2 | 0 | 0.5 |
| Coumarin | 1 | 0.4 | 0 | 0 | 0 | 0 | 0 | 0 |
| Cyclopentadecanolide | 2.6 | 3.9 | 1 | 2 | 0 | 0 | 0 | 0 |
| Decalactone Gamma | 0 | 0 | 0 | 0 | 0 | 0 | 0 | 5 |
| Ethylene Brassylate | 1 | 0 | 2 | 5 | 0 | 0 | 0 | 0 |
| Ethyl Vanillin | 0.05 | 0.05 | 0.2 | 0 | 0 | 0 | 0 | 0.03 |
| Florosa | 2.5 | 0 | 4 | 0 | 0 | 0 | 1 | 0 |
| Geraniol | 2 | 0 | 0 | 3.5 | 0.8 | 0.2 | 0 | 0 |
| Geranium Oil | 0.5 | 0 | 1 | 0.3 | 0.15 | 0 | 1 | 0 |
| Habanolide | 0 | 0 | 1 | 3 | 0 | 0 | 1 | 0 |
| Heliotropin | 0 | 0 | 6 | 0.4 | 0 | 0 | 0 | 0 |
| Hexyl Salicylate | 0 | 4 | 0 | 8.2 | 0 | 0 | 0 | 0 |
| Iso Eugenyl Acetate | 0.2 | 0 | 0 | 0 | 0 | 0 | 0 | 0 |
| Lily aldehyde | 5.5 | 6 | 0 | 5 | 0.4 | 5.4 | 1.4 | 1 |
| Linalool | 0 | 7.5 | 4 | 0 | 2 | 1 | 12 | 16 |
| Lyral | 0 | 0.7 | 0 | 0 | 0.6 | 2.6 | 0 | 0 |
| Mayol | 0 | 1 | 0 | 0.8 | 0 | 0 | 0 | 0 |
| Methyl | 5 | 0 | 0 | 17.6 | 9.6 | 0.3 | 0 | 8 |
| Dihydrojasmonate | | | | | | | | |
| Methyl Ionone Alpha | 2.5 | 0.2 | 4 | 0 | 2.8 | 0 | 4 | 0 |
| Iso Super | | | | | | | | |
| Phenyl Ethyl Acetate | 2 | 0 | 0.5 | 0 | 0 | 0 | 0 | 0 |
| Phenyl Ethyl Alcohol | 7 | 1 | 7 | 7.1 | 1.6 | 0.5 | 2 | 0 |
| Tetrahydrolinalool | 9.6 | 0 | 0 | 0.5 | 2.2 | 0 | 0 | 0 |
| Orange Oil | 0 | 0.71 | 1.125 | 0 | 0 | 6.48 | 0 | 0 |
| Bergamot Oil | 0 | 0 | 0 | 0 | 0 | 0.8 | 0 | 0 |
| Inactive ingredients, | QSP 100% | | | | QSP 100% | | | |
| solvents, excipients | | | | | | | | |
| Total Group A/% | 10.81 | 11.78 | 13.38 | 43.98 | 9.60 | 0.34 | 2.53 | 8.03 |
| Total Group B/% | 37.22 | 33.22 | 49.25 | 22.65 | 19.15 | 22.61 | 33.46 | 30.70 |
| Inactive Ingredients | 51.97 | 55 | 37.37 | 33.37 | 71.25 | 77.05 | 64.01 | 61.27 |
| Number Group A | 6 | 4 | 6 | 7 | 1 | 1 | 2 | 2 |
| Number Group B | 13 | 11 | 10 | 9 | 10 | 11 | 8 | 6 |

The active fragrances created according to the invention offered statistically significant benefits in the enhancement of positive tactile attributes and/or the suppression of negative tactile attributes when assessed by the user panel.

1. A perfume composition comprising from about 10% by weight of at least two ingredients selected from group A and from around 20% by weight of at least five ingredients selected from group B, wherein Group A consists of:
2-ethyl-4-(2,2,3-trimethylcyclopent-3-en-1-yl)but-2-en-1-ol, Citronellol, 2H-chromen-2-one, Cyclopentadecanolide, 1,4-Dioxacycloheptadecane-5,-17-dione, Ethyl Vanillin, oxacyclohexadec-12(13)-en-2-one, 1,3-benzodioxole-5-carbaldehyde and methyl(3-oxo-2-pentylcyclopentyl)acetate and Group B consists of:
3a,6,6,9a-tetramethyldodecahydronaphtho[2,1-{b}]furan, Amyl Salicylate, 4-(methyloxy)benzaldehyde, Benzyl Acetate, 3-[4-(1,1-dimethylethyl)phenyl]propanal, Cis-3-Hexenyl Acetate, Citronellyl Acetate, Cyclodecalactone-Gamma, 4-methyl-2-(2-methylpropyl)tetrahydro-2H-pyran-4-ol, Geraniol, Geranium Oil, Hexyl Salicylate, 2-(methyloxy)-4-[prop-1-enyl]phenyl acetate, 3-[4-(1,1-dimethylethyl)phenyl]-2-methylpropanal, Linalool, 4-(4-hydroxy-4-methylpentyl)cyclohex-3-ene-1-carbaldehyde, [4-(1-methylethyl)cyclohexyl]methanol, 3-methyl-4-(2,6,6-trimethylcyclohex-2-en-1-yl)but-3-en-2-one, Phenyl Ethyl Acetate, Phenyl Ethyl Alcohol and Tetrahydrolinalool;
with the proviso that perfume ingredients used as non-odorous or very low odour solvents or vehicles are not included in the calculation of the percentage composition.

2. A fragrance composition according to claim 1 comprising from about 10% by weight of at least two ingredients selected from Group A and from about 30% by weight of at least five ingredients selected from Group B.

3. A fragrance composition according to claim 1 comprising from about 10% to about 45% by weight of effective amounts of at least three of the fragrance materials listed in Group A, which are selected from:
about 0.1%-3% 2-ethyl-4-(2,2,3-trimethylcyclopent-3-en-1-yl)but-2-en-1-ol;
about 0.5%-12% Citronellol;
about 0.25%-2% 2H-chromen-2-one;
about 0.75%-5% Cyclopentadecanolide;
about 0.5%-15% Dioxacycloheptadecane-5,-17-dione;
about 0.035%-1% Ethyl Vanillin;
about 0.75%-5% oxacyclohexadec-12(13)-en-2-one;
about 0.5%-7.5% 1,3-benzodioxole-5-carbaldehyde;
about 5%-18% methyl(3-oxo-2-pentylcyclopentyl)acetate;

4. A fragrance composition according to claim 3 additionally comprising from about 10% to about 80% by weight of effective amounts of at least ten of the fragrance materials listed in Group B which are selected from:
about 0.1-1% 3a,6,6,9a-tetramethyldodecahydronaphtho[2,1-{b}]furan;
about 0.1-15% Amyl Salicylate;
about 2-7.5% 4-(methyloxy)benzaldehyde;
about 1-20% Benzyl Acetate;
about 0.5-3% 3-[4-(1,1-dimethylethyl)phenyl]propanal;
about 0.1-1% Cis-3 Hexenyl Acetate;
about 0.5-10% Cironellyl Acetate;
about 0.3-2% Cyclodecalactone Gamma;
about 2-10% 4-methyl-2-(2-methylpropyl)tetrahydro-2H-pyran-4-ol;
about 1-30% Geraniol;
about 0.25-5% Geranium Oil;
about 2-20% Hexyl Salicylate
about 0.2-2% 2-(methyloxy)-4-[prop-1-enyl]phenyl acetate;
about 4-20% 3-[4-(1,1-dimethylethyl)phenyl]-2-methylpropanal;
about 3-20% Linalool;
about 0.5-10% 4-(4-hydroxy-4-methylpentyl)cyclohex-3-ene-1-carbaldehyde;
about 0.5-2% [4-(1-methylethyl)cyclohexyl]methanol;
about 0.2-15% 3-methyl-4-(2,6,6-trimethylcyclohex-2-en-1-yl)but-3-en-2-one;
about 0.5-5% Phenyl Ethyl Acetate;
about 1-30% Phenyl Ethyl Alcohol;
about 0.5-10% Tetrahydrolinalool

6. A solid, liquid or semi-solid consumer composition for application to skin, hair, hard surfaces or fabrics, comprising a fragrance composition according to claim 1.

7. A fabric treatment composition comprising a fragrance composition according to claim 1.

8. A personal product composition comprising a fragrance composition according to claim 1.

9. A hard surface-cleaning composition comprising a fragrance composition according to claim 1.

10. A method of delivering enhanced positive tactile perception of consumer compositions adapted for application to skin, hair, hard surfaces or fabrics comprising the step of:
administering to a subject a fragrance composition according to claim 1.

11. A method for the promotion of positive tactile perception of substrates treated with consumer compositions adapted for application to skin, hair, hard surfaces or fabrics comprising the step of:
administering to a subject a fragrance formulation according to claim 1.

12. A method of delivering suppressed negative tactile perception of consumer compositions adapted for application to skin, hair, hard surfaces or fabrics comprising the step of:
administering to a subject a fragrance formulation according to claim 1.

13. A method for the suppression of negative tactile perception of consumer product compositions adapted for application to skin, hair, hard surfaces or fabrics comprising the step of:
administering to a subject a fragrance formulation according to claim 1.
